(19) United States
(12) Patent Application Publication
(10) Pub. No.: US 2017/0363597 A1
(43) Pub. Date: Dec. 21, 2017

(54) GAS SENSOR

(71) Applicant: NGK INSULATORS, LTD., Nagoya-shi (JP)

(72) Inventors: Yosuke ADACHI, Nagoya-shi (JP); Tetsuya ISHIKAWA, Kasugai-shi (JP); Jumpei TANAKA, Toyohashi-shi (JP)

(21) Appl. No.: 15/623,423

(22) Filed: Jun. 15, 2017

(30) Foreign Application Priority Data
Jun. 17, 2016 (JP) 2016-121007

(51) Int. Cl. G01N 33/00

(52) U.S. Cl. CPC G01N 33/0009

(57) ABSTRACT

A gas sensor 100 includes a sensor element 110 having a gas inlet 111; an inner protective cover 130 which has a sensor element chamber 124 thereinside and in which at least one element-chamber inlet 127 and at least one element-chamber outlet are arranged; and an outer protective cover 140 in which at least one outer inlet 144a and at least one outer outlet are arranged. A minimum path length P from the outer inlet 144a to the gas inlet 111 is 5.0 mm or more and 11.0 mm or less.

### BACKGROUND OF THE INVENTION

### 1. Field of the Invention

The present invention relates to a gas sensor.

### 2. Description of the Related Art

An example of a known gas sensor detects the concentration of predetermined gas, such as NOx or oxygen, in measurement-object gas, such as exhaust gas of an automobile. For example, PTL 1 describes a gas sensor including an outer protective cover and an inner protective cover. The inner protective cover has a cylindrical shape with a bottom and is disposed between the outer protective cover and a sensor element so as to cover the front end of the sensor element. According to PTL 1, the inner protective cover is formed in a predetermined shape so that the sensor element has quick responsiveness in gas concentration detection and high heat retaining properties at the same time.

### CITATION LIST

### Patent Literature

PTL 1: WO 2014/192945

### SUMMARY OF THE INVENTION

It is desirable that such a gas sensor has quick responsiveness in gas concentration detection.

The present invention has been made to solve the above-described problem, and the main object of the present invention is to increase the responsiveness in gas concentration detection.

To achieve the above-described object, the present invention employs the following configuration.

A gas sensor according to the present invention comprises:

a sensor element having a gas inlet through which measurement-object gas is introduced and capable of detecting a concentration of a predetermined gas in the measurement-object gas that flows into the sensor element through the gas inlet;

an inner protective cover that has a sensor element chamber thereinside and in which one or more element-chamber inlet and one or more element-chamber outlet are arranged, the sensor element chamber accommodating a front end of the sensor element and the gas inlet, the element-chamber inlet being an entrance to the sensor element chamber, and the element-chamber outlet being an exit from the sensor element chamber; and

an outer protective cover that is disposed outside the inner protective cover and in which one or more outer inlet and one or more outer outlet are arranged, the outer inlet being an entrance from outside for the measurement-object gas, and the outer outlet being an exit to the outside for the measurement-object gas,

wherein the outer protective cover and the inner protective cover form a first gas chamber and a second gas chamber as spaces therebetween, the first gas chamber being at least a portion of a flow channel for the measurement-object gas between the outer inlet and the element-chamber inlet, and the second gas chamber being at least a portion of a flow channel for the measurement-object gas between the outer outlet and the element-chamber outlet and not being directly connected to the first gas chamber, and

a minimum path length P from the outer inlet to the gas inlet is 5.0 mm or more and 11.0 mm or less.

The measurement-object gas that flows around the gas sensor enters the gas sensor through the outer inlet in the outer protective cover, passes through the first gas chamber and the element-chamber inlet, and reaches the gas inlet in the sensor element chamber. When the minimum path length P from the outer inlet to the gas inlet is 11.0 mm or less, the measurement-object gas that has entered through the outer inlet reaches the gas inlet in a relatively short time. Accordingly, the responsiveness in gas concentration detection increases. When the minimum path length P is 5.0 mm or more, the occurrence of problems due to insufficient minimum path length P can be reduced. Such problems include, for example, the risk that external poisoning materials and water that have entered through the outer inlet will easily reach the sensor element, and the risk that the sensor element will be easily cooled by the measurement-object gas.

In the gas sensor according to the present invention, the minimum path length P is preferably 10.5 mm or less, more preferably 10.0 mm or less, still more preferably less than 10.0 mm, even more preferably 9.5 mm or less, and further more preferably 9.0 mm or less. As the minimum path length P decreases, the responsiveness in gas concentration detection increases. The minimum path length P may be 7.0 mm or more, or 8.0 mm or more.

In the gas sensor according to the present invention, a cross-sectional area ratio S1/S2, which is a ratio of a total cross-sectional area S1 [mm²] of the outer inlet to a total cross-sectional area S2 [mm²] of the outer outlet, may be more than 2.0 and 5.0 or less. When the cross-sectional area ratio S1/S2 is more than 2.0, the total cross-sectional area S1 is relatively large, so that the flow rate at which the measurement-object gas enters through the outer inlet tends to increase. In addition, the total cross-sectional area S2 is relatively small, so that the flow rate at which the measurement-object gas tries to enter through the outer outlet (backflow) tends to decrease. Accordingly, the measurement-object gas in the space around the gas inlet is easily replaced by the measurement-object gas that has entered. As a result, the responsiveness in gas concentration detection increases. When the total cross-sectional area S2 is too small, the flow rate at which the measurement-object gas flows out through the outer outlet decreases, and the responsiveness may decrease accordingly. However, when the cross-sectional area ratio S1/S2 is 5.0 or less, the reduction in responsiveness can be suppressed.

In the gas sensor according to the present invention, the cross-sectional area ratio S1/S2 is preferably 2.5 or more, more preferably, 3.0 or more, and still more preferably, 3.4 or more. As the cross-sectional area ratio S1/S2 increases, the responsiveness in gas concentration detection tends to increase.

In the gas sensor according to the present invention, the total cross-sectional area S1 may be 10 mm² or more. The total cross-sectional area S1 may also be 30 mm² or less. The total cross-sectional area S2 may be 2 mm² or more. The total cross-sectional area S2 may also be 10 mm² or less.

In the gas sensor according to the present invention, the outer protective cover may have a cylindrical shape and include a side portion and a bottom portion. The outer outlet may not be arranged in the side portion of the outer protective cover. When there is an outer outlet formed in the side portion of the outer protective cover, the responsiveness may vary depending on the relationship between the position of the outer outlet in the side portion and the direction in which the measurement-object gas flows around the outer outlet. For example, when the outer outlet in the side portion opens parallel to, and toward the upstream side of, the direction in which the measurement-object gas flows, the flow of the measurement-object gas that tries to flow out from the space inside the outer protective cover through the outer outlet in the side portion is impeded by the measurement-object gas that flows around the outer outlet, and the responsiveness tends to decrease as a result. If the responsiveness greatly varies depending on the relationship between the position of the outer outlet in the side portion and the direction in which the measurement-object gas flows, the responsiveness may be reduced depending on, for example, the orientation in which the gas sensor is attached. When the outer outlet is not formed in the side portion, the influence of the orientation in which the gas sensor is attached on the responsiveness can be reduced. In this case, the outer outlet may be formed in at least one of the bottom portion and a corner portion between the side portion and the bottom portion. The outer outlet may be formed only in the bottom portion or only in the corner portion.

In the gas sensor according to the present invention, the outer protective cover may include a body portion, which has a cylindrical shape and in which the outer inlet is arranged, and a front end portion, which has a cylindrical shape with a bottom and an inner diameter smaller than an inner diameter of the body portion and in which the outer outlet is arranged, the front end portion being located in front of the body portion in a forward direction, which is a direction from a back end toward the front end of the sensor element. The outer protective cover and the inner protective cover may form the first gas chamber as a space between the body portion of the outer protective cover and the inner protective cover, and the second gas chamber as a space between the front end portion of the outer protective cover and the inner protective cover.

In the gas sensor according to the present invention, the element-chamber inlet may be formed in the inner protective cover so that an element-side opening of the element-chamber inlet that is close to the sensor element chamber opens in a forward direction, which is a direction from a back end toward the front end of the sensor element. In this case, the measurement-object gas that has flowed out through the element-side opening is not blown against a surface of the sensor element (surface other than the gas inlet) in a direction perpendicular to the surface of the sensor element, nor does it flow a long distance along the surface of the sensor element before reaching the gas inlet. Accordingly, cooling of the sensor element can be reduced. Cooling of the sensor element is reduced by adjusting the direction in which the element-side opening opens, and not by reducing the flow rate and flow velocity of the measurement-object gas inside the inner protective cover. Therefore, the amount of reduction in the responsiveness in gas concentration detection can be reduced. As a result, the sensor element has quick responsiveness and high heat retaining properties at the same time. Here, the phrase “the element-side opening opens in the forward direction” includes a case in which the element-side opening opens parallel to the forward direction of the sensor element and a case in which the element-side opening opens obliquely to the forward direction so as to become closer to the sensor element with increasing distance in the forward direction of the sensor element.

In the gas sensor according to the present invention, the inner protective cover may include a first member and a second member, and the element-chamber inlet may be formed as a gap between the first member and the second member. Also, the first member may include a first cylindrical portion that surrounds the sensor element, and the second member may include a second cylindrical portion having a diameter larger than a diameter of the first cylindrical portion. The element-chamber inlet may be a tubular gap between an outer peripheral surface of the first cylindrical portion and an inner peripheral surface of the second cylindrical portion.

### BRIEF DESCRIPTION OF THE DRAWINGS

FIG. 1 is a schematic diagram illustrating the manner in which a gas sensor 100 is attached to a pipe 20.

FIG. 2 is a sectional view taken along line A-A in FIG. 1.

FIG. 3 is a sectional view taken along line B-B in FIG. 2.

FIG. 4 is a sectional view taken along line C-C in FIG. 3.

FIG. 5 is a sectional view of an outer protective cover 140 taken along line C-C in FIG. 3.

FIG. 6 is a view in the direction of arrow D in FIG. 3.

FIG. 7 is an enlarged partial sectional view taken along line E-E in FIG. 4.

FIG. 8 is a sectional view illustrating the case in which outer outlets 147a include a plurality of horizontal holes 147b.

FIG. 9 is a perspective view illustrating the case in which the outer outlets 147a include a plurality of horizontal holes 147b.

FIG. 10 is a sectional view illustrating the case in which the outer outlets 147a include corner holes 147d.

FIG. 11 is a sectional view illustrating element-chamber inlets 227 according to a modification.

FIG. 12 is a vertical sectional view of a gas sensor 300 according to a modification.

FIG. 13 is a sectional view of an outer protective cover 140 according to Experimental Example 4.

FIG. 14 is an enlarged partial sectional view of a gas sensor 100 according to Experimental Example 5.

FIG. 15 is a graph showing the angular dependence of the response time of gas sensors according to Experimental Examples 1 to 5.

FIG. 16 is a graph showing the relationship between the flow velocity V and the response time of Experimental Examples 1 to 5.

### DETAILED DESCRIPTION OF THE INVENTION

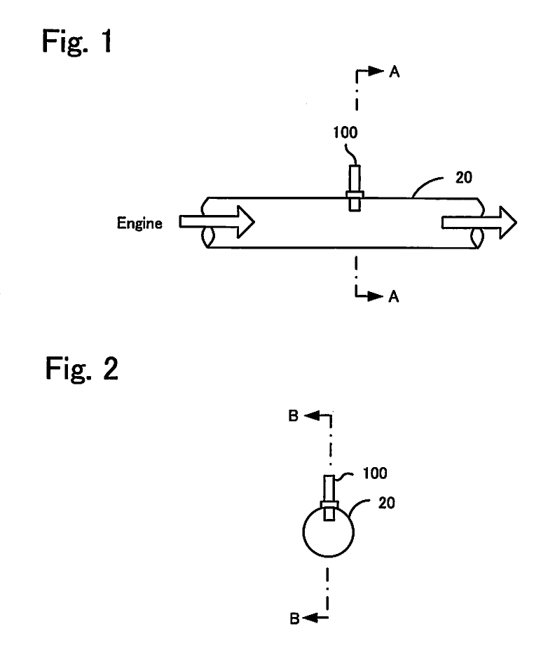

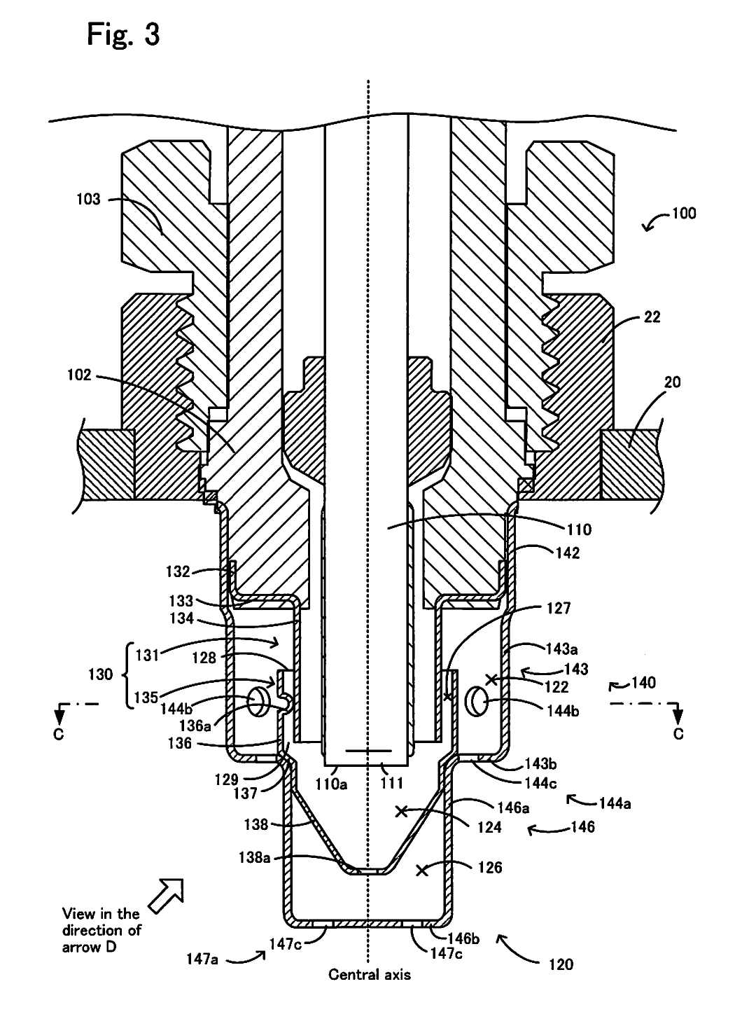

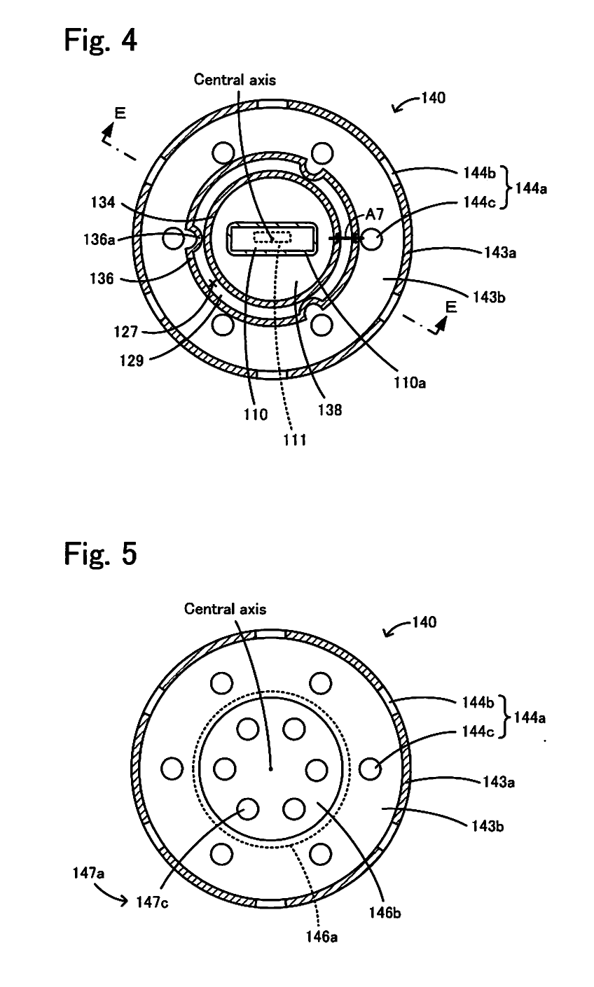

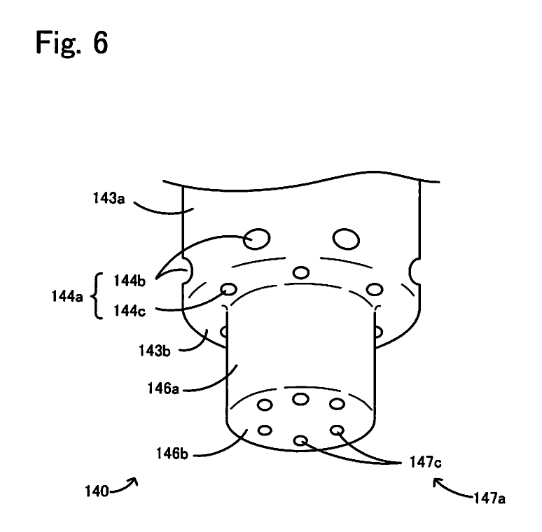

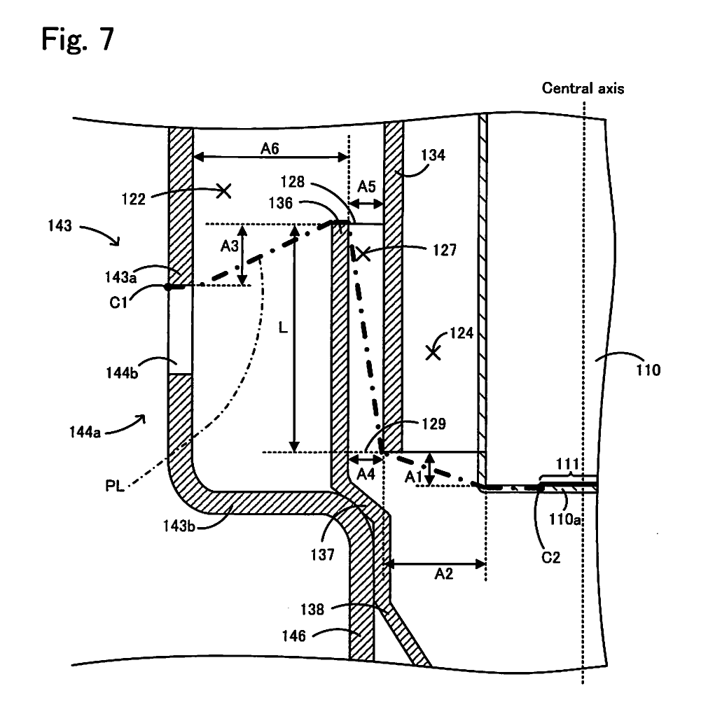

Embodiments of the present invention will now be described with reference to the drawings. FIG. 1 is a schematic diagram illustrating the manner in which a gas sensor 100 is attached to a pipe 20. FIG. 2 is a sectional view taken along line A-A in FIG. 1. FIG. 3 is a sectional view taken along line B-B in FIG. 2. FIG. 4 is a sectional view taken along line C-C in FIG. 3. FIG. 5 is a sectional view of an outer protective cover 140 taken along line C-C in FIG. 3. FIG. 5 illustrates the structure in which a first cylindrical portion 134, a second cylindrical portion 136, a front end portion 138, and a sensor element 110 are removed from the structure illustrated in FIG. 4. FIG. 6 is a view in the direction of arrow D in FIG. 3. FIG. 7 is an enlarged partial sectional view taken along line E-E in FIG. 4.

As illustrated in FIG. 1, the gas sensor 100 is attached to the pipe 20, which is an exhaust path from an engine of a vehicle. The gas sensor 100 detects the concentration of at least one of gas components, such as NOx and O₂, of exhaust gas that is discharged from the engine as measurement-object gas. As illustrated in FIG. 2, the gas sensor 100 is fixed to the pipe 20 so that the central axis thereof is perpendicular to the flow of the measurement-object gas in the pipe 20. Note that the gas sensor 100 may be fixed to the pipe 20 so that the central axis thereof is perpendicular to the flow of the measurement-object gas in the pipe 20 and at a predetermined angle (for example, 45°) with respect to the vertical direction.

As illustrated in FIG. 3, the gas sensor 100 includes a sensor element 110 having a function of detecting the concentration of predetermined gas in the measurement-object gas, and a protective cover 120 that protects the sensor element 110. The gas sensor 100 also includes a metal housing 102 and a metal nut 103 having an external thread on an outer peripheral surface thereof. The housing 102 is inserted through a fixing member 22 that is welded to the pipe 20 and that has an internal screw on an inner peripheral surface thereof, and the nut 103 is screwed into the fixing member 22 so that the housing 102 is fixed to the fixing member 22. Thus, the gas sensor 100 is fixed to the pipe 20. The direction in which the measurement-object gas flows through the pipe 20 is the left-to-right direction in FIG. 3.

The sensor element 110 is a thin elongated plate-shaped element, and has a multilayer structure including a plurality of layers of oxygen ion conductive solid electrolyte, such as zirconia (ZrO₂). The sensor element 110 has a gas inlet 111 through which the measurement-object gas is introduced, and is capable of detecting the concentration of the predetermined gas (for example, NOx or O₂) in the measurement-object gas that flows into the sensor element 110 through the gas inlet 111. In the present embodiment, the gas inlet 111 opens in the front end face of the sensor element 110 (bottom surface of the sensor element 110 in FIG. 3). The sensor element 110 has a heater disposed therein, the heater having a function of heating the sensor element 110 to adjust the temperature thereof. The structure of the sensor element 110 and the principle of gas concentration detection are commonly known, and are described in, for example, Japanese Unexamined Patent Application Publication No. 2008-164411. The front end (bottom end in FIG. 3) and the gas inlet 111 of the sensor element 110 are disposed in a sensor element chamber 124. The direction from the back end toward the front end of the sensor element 110 (downward direction in FIG. 3) is referred to as a forward direction.

The sensor element 110 includes a porous protective layer 110a that at least partially covers the surface thereof. In the present embodiment, the porous protective layer 110a is formed on five of the six faces of the sensor element 110, and covers substantially the entire surface of a portion of the sensor element 110 that is exposed in the sensor element chamber 124. More specifically, the porous protective layer 110a covers the entirety of the front end face (bottom face in FIG. 3) of the sensor element 110 in which the gas inlet 111 is formed. In addition, the porous protective layer 110a covers four faces (top, bottom, left and right faces in FIG. 4) of the sensor element 110 that are connected to the front end face of the sensor element 110 over areas near the front end face of the sensor element 110. The porous protective layer 110a has a function of, for example, suppressing formation of cracks in the sensor element 110 due to adhesion of water or the like contained in the measurement-object gas. The porous protective layer 110a also has a function of suppressing adhesion of an oil component or the like contained in the measurement-object gas to electrodes (not shown) on the surface of the sensor element 110. The porous protective layer 110a may be formed of a porous material, such as an alumina porous material, a zirconia porous material, a spinel porous material, a cordierite porous material, a titania porous material, or a magnesia porous material. The porous protective layer 110a may be formed by, for example, plasma spraying, screen printing, or dipping. Although the gas inlet 111 is also covered with the porous protective layer 110a, the measurement-object gas can flow through the porous protective layer 110a and reach the gas inlet 111 because the porous protective layer 110a is formed of a porous material. The porous protective layer 110a may have a thickness of, for example, 100 μm to 700 μm; however, the thickness is not limited to this.

The protective cover 120 is disposed so as to surround the sensor element 110. The protective cover 120 includes an inner protective cover 130 that has a cylindrical shape with a bottom and that covers the front end of the sensor element 110, and an outer protective cover 140 that has a cylindrical shape with a bottom and that covers the inner protective cover 130. A first gas chamber 122 and a second gas chamber 126 are formed as spaces defined between the inner protective cover 130 and the outer protective cover 140, and the sensor element chamber 124 is formed as a space surrounded by the inner protective cover 130. The gas sensor 100, the sensor element 110, the inner protective cover 130, and the outer protective cover 140 have the same central axis. The protective cover 120 is made of a metal (for example, stainless steel).

The inner protective cover 130 includes a first member 131 and a second member 135. The first member 131 includes a large-diameter portion 132 having a cylindrical shape, a first cylindrical portion 134 having a diameter smaller than that of the large-diameter portion 132, and a step portion 133 that connects the large-diameter portion 132 and the first cylindrical portion 134. The first cylindrical portion 134 surrounds the sensor element 110. The second member 135 includes a second cylindrical portion 136 having a diameter larger than that of the first cylindrical portion 134; a front end portion 138 having an inverted truncated conical shape that is located in front of the second cylindrical portion 136 in the forward direction of the sensor element 110 (downward direction in FIG. 3); and a connection portion 137 that connects the second cylindrical portion 136 and the front end portion 138. A single element-chamber outlet 138a (also referred to as an inner gas hole) having a circular shape is formed at the center of the bottom face of the front end portion 138. The element-chamber outlet 138a is connected to the sensor element chamber 124 and the second gas chamber 126, and serves as an exit for the measurement-object gas in the sensor element chamber 124. The diameter of the element-chamber outlet 138a is not particularly limited, and may be, for example, 0.5 mm to 2.6 mm. The element-chamber outlet 138a is located in front of the gas inlet 111 in the forward direction of the sensor element 110 (downward direction in FIG. 3). In other words, the element-chamber outlet 138a is farther from the back end of the sensor element 110 (upper end (not illustrated) of the sensor element 110 in FIG. 3) than the gas inlet 111 is (below the gas inlet 111 in FIG. 3).

The large-diameter portion 132, the first cylindrical portion 134, the second cylindrical portion 136, and the front end portion 138 have the same central axis. The inner peripheral surface of the large-diameter portion 132 is in contact with the housing 102 so that the first member 131 is fixed to the housing 102. The outer peripheral surface of the connection portion 137 of the second member 135 is in contact with and fixed to the inner peripheral surface of the outer protective cover 140 by, for example, welding. The second member 135 may instead be fixed by forming the front end portion 138 so that outer diameter thereof is slightly larger than the inner diameter of a front end portion 146 of the outer protective cover 140 and press-fitting the front end portion 138 into the front end portion 146.

A plurality of protruding portions 136a are formed on the inner peripheral surface of the second cylindrical portion 136 so as to protrude toward and be in contact with the outer peripheral surface of the first cylindrical portion 134. As illustrated in FIG. 4, three protruding portions 136a are arranged at equal intervals in the circumferential direction of the inner peripheral surface of the second cylindrical portion 136. The protruding portions 136a have a substantially hemispherical shape. Since the protruding portions 136a are provided, the positional relationship between the first cylindrical portion 134 and the second cylindrical portion 136 can be easily fixed by the protruding portions 136a. The protruding portions 136a preferably press the outer peripheral surface of the first cylindrical portion 134 radially inward. In such a case, the positional relationship between the first cylindrical portion 134 and the second cylindrical portion 136 can be more reliably fixed by the protruding portions 136a. The number of protruding portions 136a is not limited to three, and may instead be two, or four or more. Preferably, three or more protruding portions 136a are provided so that the first cylindrical portion 134 and the second cylindrical portion 136 can be stably fixed.

An element-chamber inlet 127 (see FIGS. 3, 4, and 7) is formed in the inner protective cover 130. The element-chamber inlet 127 is a gap between the first member 131 and the second member 135, and serves as an entrance to the sensor element chamber 124 for the measurement-object gas. More specifically, the element-chamber inlet 127 is a tubular gap (gas flow channel) between the outer peripheral surface of the first cylindrical portion 134 and the inner peripheral surface of the second cylindrical portion 136. The element-chamber inlet 127 includes an outer opening 128 and an element-side opening 129. The outer opening 128 is an opening adjacent to the first gas chamber 122, which is a space in which outer inlets 144a are arranged. The element-side opening 129 is an opening adjacent to the sensor element chamber 124, which is a space in which the gas inlet 111 is arranged. The outer opening 128 is closer to the back end of the sensor element 110 (upper end in FIG. 3) than the element-side opening 129 is. Therefore, in the path of the measurement-object gas from the outer inlets 144a to the gas inlet 111, the element-chamber inlet 127 serves as a flow channel extending from the back-end side (upper side in FIG. 3) toward the front-end side (lower side in FIG. 3) of the sensor element 110. Also, the element-chamber inlet 127 is a flow channel that is parallel to the front-back direction of the sensor element 110 (vertical flow channel in FIG. 3).

The element-side opening 129 is preferably located so that the distance A1 from the gas inlet 111 (see FIG. 7) is −1.5 mm or more. The distance A1 may be 0 mm or more, or more than 1.5 mm. The distance A1 is the distance in the front-back direction of the sensor element 110 (vertical direction in FIG. 3), and the front-to-back direction (upward direction in FIG. 3) is defined as positive. More specifically, the distance A1 is the distance between a portion of the opening edge of the gas inlet 111 that is closest to the element-side opening 129 and a portion of the edge of the element-side opening 129 that is closest to the gas inlet 111 in the front-back direction of the sensor element 110. In FIG. 3, if the gas inlet is a horizontal hole that opens in a side surface of the sensor element 110, and if the element-side opening 129 is located between the top and bottom ends of the opening of the gas inlet, the distance A1 is defined as 0 mm. The upper limit of the distance A1 is determined by the shapes of the inner protective cover 130 and the sensor element chamber 124. Although there is no particular limitation, the distance A1 may be 7.5 mm or less, 5 mm or less, or 2 mm or less.

The element-side opening 129 is located at a distance A2 (see FIG. 7) from the sensor element 110. The distance A2 is the distance in a direction perpendicular to the front-back direction of the sensor element 110. More specifically, the distance A2 is the distance between a portion of the sensor element 110 that is closest to the element-side opening 129 and a portion of the edge of the element-side opening 129 that is closest to the sensor element 110 in the direction perpendicular to the front-back direction of the sensor element 110. As the distance A2 increases, the element-side opening 129 becomes farther away from the sensor element 110, so that cooling of the sensor element 110 can be further reduced. The distance A2 is not particularly limited, and may be, for example, 0.6 mm to 3.0 mm. The element-side opening 129 opens parallel to the front-back direction of the sensor element 110 in the back-to-front direction of the sensor element 110. In other words, the element-side opening 129 opens downward (toward the region directly below) in FIGS. 3 and 7. Thus, the sensor element 110 is disposed outside the region to which the element-chamber inlet 127 is virtually extended from the element-side opening 129 (region directly below the element-side opening 129 in FIGS. 3 and 7). Accordingly, the measurement-object gas that flows out through the element-side opening 129 is not directly blown against the surface of the sensor element 110, and cooling of the sensor element 110 can be reduced.

The outer opening 128 is located at a distance A3 from the outer inlet 144a (see FIG. 7). The distance A3 is the distance in the front-back direction of the sensor element 110 (vertical direction in FIGS. 3 and 7). Similar to the distance A1, the front-to-back direction is defined as positive. More specifically, the distance A3 is the distance between a portion of the opening edge of the outer inlet 144a that is closest to the outer opening 128 and a portion of the edge of the outer opening 128 that is closest to the outer inlet 144a in the front-back direction of the sensor element 110. In the present embodiment, a plurality of outer inlets 144a including horizontal holes 144b and vertical holes 144c are provided, and the upper ends of the horizontal holes 144b are closest to the outer opening 128 in the vertical direction in FIG. 3. Therefore, referring to FIG. 7, the distance A3 is the distance between the upper end of the horizontal hole 144b and the outer opening 128. When, for example, the outer opening 128 is below the lower end of the vertical hole 144c in the vertical direction in FIG. 3, the distance A3 is the distance between the lower end of the vertical hole 144c and the outer opening 128 in the vertical direction. The outer opening 128 may be located so that the distance A3 is 0 or more, or positive. Alternatively, the outer opening 128 may be located so that the distance A3 is 0 or less, or negative. The distance A3 is not particularly limited, and may be, for example, −3 mm or more and 3 mm or less. Alternatively, the distance A3 may be −2 mm or more, −1 mm or more, 2 mm or less, or 1 mm or less.

The outer opening 128 is located at a distance A6 from the outer inlet 144a (see FIG. 7). The distance A6 is the distance in the direction perpendicular to the front-back direction of the sensor element 110 (vertical direction in FIGS. 3 and 7). The distance A6 is the distance between the outer inlet 144a that is closest to the outer opening 128 in the front-back direction of the sensor element 110 and the outer opening 128. In the present embodiment, the distance A6 is equal to one-half the difference between the inner diameter of a side portion 143a and the inner diameter of the second cylindrical portion 136. The distance A6 is not particularly limited, and may be, for example, more than 0 mm and 2.5 mm or less. Alternatively, the distance A6 may be 0.5 mm or more, 1 mm or more, 2.0 mm or less, or 1.5 mm or less.

The outer peripheral surface of the first cylindrical portion 134 and the inner peripheral surface of the second cylindrical portion 136 are apart from each other in the radial direction of the first and second cylindrical portions 134 and 136 by a distance A4 at the element-side opening 129, and by a distance A5 at the outer opening 128. The outer peripheral surface of the first cylindrical portion 134 and the inner peripheral surface of the second cylindrical portion 136 are apart from each other by a distance A7 at a location where the protruding portions 136a are in contact with the first cylindrical portion 134 (location of the sectional view of FIG. 4). The distances A4, A5, and A7 are not particularly limited, and may be, for example, 0.3 mm to 2.4 mm. The opening areas of the element-side opening 129 and the outer opening 128 can be adjusted by adjusting the distances A4 and A5. In the present embodiment, the distances A4, A5, and A7 are equal, and the element-side opening 129 and the outer opening 128 have the same opening area. In the present embodiment, the distance A4 (distances A5 and A7) is equal to one-half the difference between the outer diameter of the first cylindrical portion 134 and the inner diameter of the second cylindrical portion 136. The distance between the element-side opening 129 and the outer opening 128 in the vertical direction, that is, the length L of the element-chamber inlet 127 in the vertical direction (which corresponds to the path length of the element-chamber inlet 127), is not particularly limited, and may be, for example, more than 0 mm and 6.6 mm or less. Alternatively, the length L may be 3 mm or more, or 5 mm or less.

As illustrated in FIG. 3, the outer protective cover 140 includes a large-diameter portion 142 that has a cylindrical shape; a body portion 143 that has a cylindrical shape, that is connected to the large-diameter portion 142, and whose diameter is smaller than that of the large-diameter portion 142; and the front end portion 146 that has a cylindrical shape with a bottom and whose inner diameter is smaller than that of the body portion 143. The body portion 143 includes the side portion 143a, which has a side surface that extends in the direction of the central axis of the outer protective cover 140 (vertical direction in FIG. 3), and a step portion 143b that defines the bottom of the body portion 143 and connects the side portion 143a and the front end portion 146. The central axes of the large-diameter portion 142, the body portion 143, and the front end portion 146 coincide with the central axis of the inner protective cover 130. The inner peripheral surface of the large-diameter portion 142 is in contact with the housing 102 and the large-diameter portion 132, so that the outer protective cover 140 is fixed to the housing 102. The body portion 143 is arranged so as to cover the outer peripheries of the first cylindrical portion 134 and the second cylindrical portion 136. The front end portion 146 is arranged so as to cover the front end portion 138, and the inner peripheral surface thereof is in contact with the outer peripheral surface of the connection portion 137. The front end portion 146 includes a side portion 146a, which has a side surface that extends in the direction of the central axis of the outer protective cover 140 (vertical direction in FIG. 3) and whose outer diameter is smaller than the inner diameter of the side portion 143a, and a bottom portion 146b that defines the bottom of the outer protective cover 140. The front end portion 146 is located in front of the body portion 143 in the forward direction. The outer protective cover 140 has a plurality of outer inlets 144a (twelve outer inlets 144a in the present embodiment) formed in the body portion 143 and a plurality of outer outlets 147a (six outer outlets 147a in the present embodiment) formed in the front end portion 146. The outer inlets 144a are entrances from the outside for the measurement-object gas, and the outer outlets 147a are exits to the outside for the measurement-object gas.

The outer inlets 144a are holes (referred to also as first outer gas holes) that connect the region outside the outer protective cover 140 (the outside) to the first gas chamber 122. The outer inlets 144a include a plurality of horizontal holes 144b (six horizontal holes 144b in the present embodiment) formed in the side portion 143a at equal intervals therebetween and a plurality of vertical holes 144c (six vertical holes 144c in the present embodiment) formed in the step portion 143b at equal intervals therebetween (see FIGS. 3 to 6). The outer inlets 144a (horizontal holes 144b and vertical holes 144c) are circular (perfect circular) holes. The diameters of the twelve outer inlets 144a are not particularly limited, and may be, for example, 0.5 mm to 2 mm. Alternatively, the diameters of the outer inlets 144a may be 1.5 mm or less. In the present embodiment, the horizontal holes 144b have the same diameter, and the vertical holes 144c have the same diameter. The diameter of the horizontal holes 144b is larger than that of the vertical holes 144c. As illustrated in FIGS. 4 and 5, the outer inlets 144a are formed so that the horizontal holes 144b and the vertical holes 144c are alternately arranged at equal intervals in the circumferential direction of the outer protective cover 140. In other words, in FIGS. 4 and 5, the line connecting the central axis of the outer protective cover 140 and the center of any horizontal hole 144b and the line connecting the central axis of the outer protective cover 140 and the center of one of the vertical holes 144c that is adjacent to that horizontal hole 144b form an angle of 30° (360°/12).

The outer outlets 147a are holes (referred to also as second outer gas holes) that connect the region outside the outer protective cover 140 (the outside) to the second gas chamber 126. The outer outlets 147a include a plurality of vertical holes 147c (six vertical holes 147c in the present embodiment) formed in the bottom portion 146b of the front end portion 146 at equal intervals therebetween in the circumferential direction of the outer protective cover 140 (see FIGS. 3, 5, and 6). Unlike the outer inlets 144a, none of the outer outlets 147a is arranged in a side portion of the outer protective cover 140 (side portion 146a of the front end portion 146 in this case). The outer outlets 147a (vertical holes 147c in this example) are circular (perfect circular) holes. The diameters of the six outer outlets 147a are not particularly limited, and may be, for example, 0.5 mm to 2.0 mm. Alternatively, the diameters of the outer outlets 147a may be 1.5 mm or less. In the present embodiment, the outer outlets 147a have the same diameter. The diameter of the vertical holes 147c is smaller than the diameter of the horizontal holes 144b.

The outer protective cover 140 and the inner protective cover 130 form the first gas chamber 122 as a space between the body portion 143 and the inner protective cover 130. More specifically, the first gas chamber 122 is a space surrounded by the step portion 133, the first cylindrical portion 134, the second cylindrical portion 136, the large-diameter portion 142, the side portion 143a, and the step portion 143b. The sensor element chamber 124 is a space surrounded by the inner protective cover 130. The outer protective cover 140 and the inner protective cover 130 also form the second gas chamber 126 as a space between the front end portion 146 and the inner protective cover 130. More specifically, the second gas chamber 126 is a space surrounded by the front end portion 138 and the front end portion 146. Since the inner peripheral surface of the front end portion 146 is in contact with the outer peripheral surface of the connection portion 137, the first gas chamber 122 and the second gas chamber 126 are not directly connected to each other.

The manner in which the measurement-object gas flows inside the protective cover 120 when the gas sensor 100 detects the concentration of the predetermined gas will now be described. First, the measurement-object gas that flows through the pipe 20 enters the first gas chamber 122 through at least one of the outer inlets 144a (horizontal holes 144b and vertical holes 144c). Next, the measurement-object gas enters the element-chamber inlet 127 from the first gas chamber 122 through the outer opening 128, flows through the element-chamber inlet 127, and enters the sensor element chamber 124 through the element-side opening 129. At least part of the measurement-object gas that has entered the sensor element chamber 124 through the element-side opening 129 reaches the gas inlet 111 of the sensor element 110. When the measurement-object gas reaches the gas inlet 111 and enters the sensor element 110, the sensor element 110 generates an electrical signal (voltage or current) corresponding to the concentration of the predetermined gas (for example, NOx or O₂) in the measurement-object gas. The gas concentration is detected on the basis of this electrical signal. The measurement-object gas in the sensor element chamber 124 enters the second gas chamber 126 through the element-chamber outlet 138a, and flows out through at least one of the outer outlets 147a. The output of the heater disposed in the sensor element 110 is controlled by, for example, a controller (not shown) so that the temperature of the sensor element 110 is maintained at a predetermined temperature.

The protective cover 120 is preferably formed so that, when the measurement-object gas flows inside the protective cover 120 in the above-described manner, a minimum path length P from the outer inlets 144a to the gas inlet 111 is 5.0 mm or more and 11.0 mm or less. In the present embodiment, the minimum path length P is the length of the broken line PL, that is, the bold one-dot chain line, in FIG. 7. The minimum path length P is the length of the shortest path for the measurement-object gas from the outer opening of the outer inlet 144a to the outer opening of the gas inlet 111. When there is a plurality of outer inlets 144a, the minimum path length P is the shortest one of the minimum path lengths from the outer inlets 144a to the gas inlet 111. In the present embodiment, the outer protective cover 140 has the horizontal holes 144b and the vertical holes 144c as the outer inlets 144a. As illustrated in FIG. 3, the horizontal holes 144b are disposed above the vertical holes 144c, and are closer to the outer opening 128 than the vertical holes 144c. In addition, in the present embodiment, as illustrated in FIG. 4, the gas inlet 111 has a rectangular opening, and is shifted upward in FIG. 4 from the central axis of the inner protective cover 130 and the outer protective cover 140. Accordingly, in the present embodiment, the minimum path length from one of the six horizontal holes 144b that is at the upper left in FIG. 4 to the gas inlet 111 is the minimum path length P of the protective cover 120. The minimum path length from the horizontal hole 144b at the upper right in FIG. 4 to the gas inlet 111 is also the same (=minimum path length P). FIG. 7 is an enlarged partial sectional view of a region around the horizontal hole 144b at the upper left in FIG. 4 taken along line E-E. The horizontal hole 144b illustrated in FIG. 7 is the horizontal hole 144b at the upper left in FIG. 4. Referring to FIG. 7, the minimum path length P is the length of the shortest path (broken line PL) from an end portion C1 (upper end portion in FIG. 7) of the outer opening of the horizontal hole 144b, the end portion C1 being closest to the outer opening 128, to an end portion C2 (left end portion in FIG. 7) of the outer opening of the gas inlet 111. The minimum path length P is determined without considering the porous protective layer 110a. For example, in FIG. 7, a portion of the path shown by the broken line PL from the element-side opening 129 to the gas inlet 111 is determined as the combination of the straight line connecting the element-side opening 129 and the lower left corner of the sensor element 110 and the straight line connecting the lower left corner of the sensor element 110 and the left end of the opening of the gas inlet 111 without considering the porous protective layer 110a. In the present embodiment, as described above, the shape, location, etc., of the gas inlet 111 are such that the minimum path lengths from the four horizontal holes 144b other than the horizontal holes 144b at the upper left and upper right in FIG. 4 to the gas inlet 111 are slightly greater than the minimum path length P. When the horizontal holes 144b have different minimum path lengths as in this case, the minimum path lengths of as many horizontal holes 144b as possible are preferably 5.0 mm or more and 11.0 mm or less. In the present embodiment, not only the minimum path length P of the horizontal holes 144b at the upper left and upper right in FIG. 4 but also the minimum path lengths of the other horizontal holes 144b are 5.0 mm or more and 11.0 mm or less. In addition to the horizontal holes 144b, the minimum path length from at least one of the vertical holes 144c to the gas inlet 111 may also be 5.0 mm or more and 11.0 mm or less. Furthermore, the minimum path lengths from the vertical holes 144c to the gas inlet 111 may all be 5.0 mm or more and 11.0 mm or less. Furthermore, the minimum path lengths from the outer inlets 144a (horizontal holes 144b and vertical holes 144c in this case) to the gas inlet 111 may all be 5.0 mm or more and 11.0 mm or less.

The sensor element 110 included in the gas sensor 100 is preferably capable of quickly detecting a change in the concentration of the predetermined gas in the measurement-object gas. In other words, the sensor element 110 preferably has quick responsiveness in gas concentration detection. When the minimum path length P determined as described above is as small as 11.0 mm or less, the measurement-object gas that has entered through the outer inlets 144a reaches the gas inlet 111 in a relatively short time, and the responsiveness increases accordingly. When the minimum path length P is 5.0 mm or more, the occurrence of problems due to insufficient minimum path length P can be reduced. Such problems include, for example, the risk that external poisoning materials and water that have entered through the outer inlets 144a will easily reach the sensor element 110, and the risk that the sensor element 110 will be easily cooled by the measurement-object gas or the output of the heater required to prevent cooling of the sensor element 110 will be increased. The minimum path length P is preferably 10.5 mm or less, more preferably, 10.0 mm or less, still more preferably, less than 10.0 mm, still more preferably, 9.5 mm or less, and still more preferably, 9.0 mm or less. As the minimum path length P decreases, the responsiveness in gas concentration detection increases. The minimum path length P may be adjusted by, for example, adjusting at least one of the distances A1 to A7 and the length L in FIG. 7 or by adjusting the diameters of the outer inlets 144a. The minimum path length P may be 7.0 mm or more, or 8.0 mm or more.

The outer protective cover 140 is preferably structured so that a cross-sectional area ratio S1/S2, which is a ratio of the total cross-sectional area S1 [mm²] of the outer inlets 144a to the total cross-sectional area S2 [mm²] of the outer outlets 147a, is more than 2.0 and 5.0 or less. When the cross-sectional area ratio S1/S2 is more than 2.0, the total cross-sectional area S1 is relatively large, so that the flow rate at which the measurement-object gas enters through the outer inlets 144a tends to increase. In addition, the total cross-sectional area S2 is relatively small, so that the flow rate at which the measurement-object gas tries to enter through the outer outlets 147a (backflow) tends to decrease. Accordingly, the measurement-object gas in the space around the gas inlet 111 is easily replaced by the measurement-object gas that has entered. As a result, the responsiveness in gas concentration detection increases. When the total cross-sectional area S2 is too small, the flow rate at which the measurement-object gas flows out through the outer outlets 147a decreases, and the responsiveness may decrease accordingly. However, when the cross-sectional area ratio S1/S2 is 5.0 or less, the reduction in responsiveness can be suppressed. The cross-sectional area ratio S1/S2 may be adjusted by, for example, adjusting the numbers of the outer inlets 144a and the outer outlets 147a, or by adjusting the cross-sectional areas of the outer inlets 144a and the outer outlets 147a.

In the present embodiment, the total cross-sectional area S1 is the sum of the total cross-sectional area of the six horizontal holes 144b and the total cross-sectional area of the six vertical holes 144c. The total cross-sectional area S2 is the sum of the cross-sectional areas of the six vertical holes 147c. The cross-sectional area of each outer inlet 144a is the area of the outer inlet 144a along a plane perpendicular to the direction in which the measurement-object gas flows through the outer inlet 144a. In the present embodiment, the outer inlets 144a are holes having circular shapes, and the areas of the circular shapes serve as the cross-sectional areas thereof. This also applies to the outer outlets 147a. When, for example, one of the outer inlets 144a is shaped so that the cross-sectional area thereof is not constant, for example, so that the cross-sectional area thereof differs between the entrance side (outer surface of the outer protective cover 140) and the exit side (inner surface of the outer protective cover 140), the minimum value of the cross-sectional area is defined as the cross-sectional area of that outer inlet 144a. This also applies to the outer outlets 147a.

The cross-sectional area ratio S1/S2 is preferably 2.5 or more, more preferably, 3.0 or more, and still more preferably, 3.4 or more. As the cross-sectional area ratio S1/S2 increases, the responsiveness in gas concentration detection tends to increase. The total cross-sectional area S1 may be 10 mm² or more. The total cross-sectional area S1 may also be 30 mm² or less. The total cross-sectional area S2 may be 2 mm² or more. The total cross-sectional area S2 may also be 10 mm² or less.

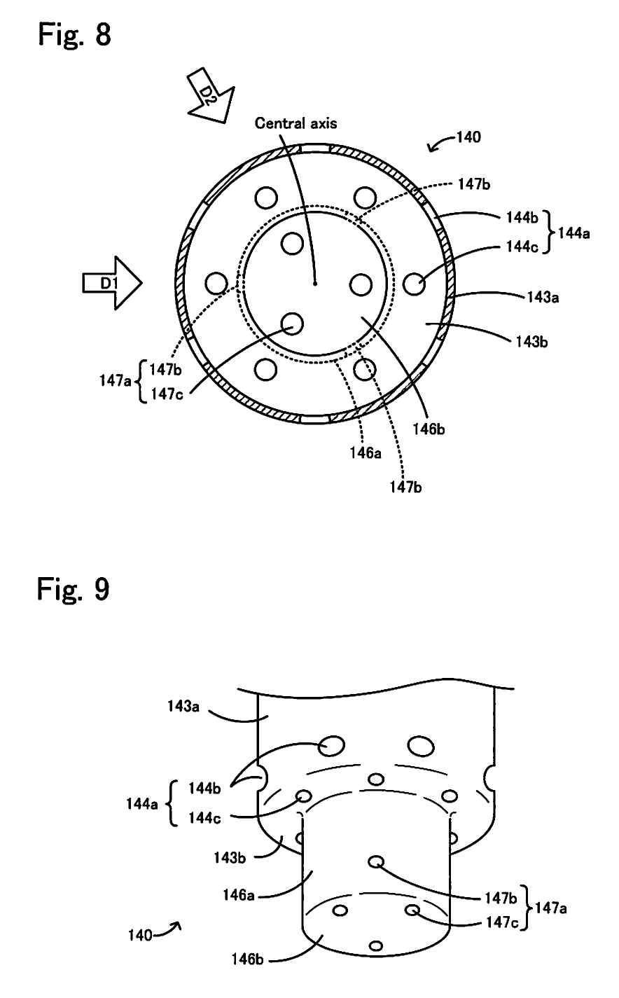

In the present embodiment, the outer protective cover 140 includes the side portion 146a and the bottom portion 146b and has a cylindrical shape with a bottom. The outer outlets 147a are not formed in the side portion 146a of the outer protective cover 140. If the outer outlets 147a are formed in the side portion 146a of the outer protective cover 140, the responsiveness may vary depending on the relationship between the positions of the outer outlets 147a in the side portion 146a and the direction in which the measurement-object gas flows around the outer outlets 147a. FIGS. 8 and 9 are a sectional view and a perspective view, respectively, illustrating the case in which the outer outlets 147a include a plurality of horizontal holes 147b (three horizontal holes 147b in this example) formed in the side portion 146a. The outer protective cover 140 illustrated in FIGS. 8 and 9 has outer outlets 147a including three horizontal holes 147b and three vertical holes 147c. The horizontal holes 147b and the vertical holes 147c are alternately arranged at equal intervals in the circumferential direction of the outer protective cover 140. In the outer protective cover 140 illustrated in FIGS. 8 and 9, when, for example, the direction in which the measurement-object gas flows is the left-to-right direction as shown by arrow D1 in FIG. 8, one of the horizontal holes 147b (the leftmost horizontal hole 147b in FIG. 8) opens parallel to, and toward the upstream side (leftward in FIG. 8) of, the direction in which the measurement-object gas flows. In this case, the flow of the measurement-object gas that tries to flow out from the space inside the outer protective cover 140 through this horizontal hole 147b is impeded by the measurement-object gas that flows around this horizontal hole 147b, and the responsiveness tends to decrease as a result. In contrast, assume that the direction in which the measurement-object gas flows is the direction shown by arrow D2 in FIG. 8. The direction of arrow D2 is the direction obtained by rotating the direction of arrow D1 clockwise by 60° in FIG. 8, and is toward the middle point between the left horizontal hole 147b and the upper right horizontal hole 147b in the side portion 146a of the outer protective cover 140 in FIG. 8. In this case, the horizontal holes 147b are arranged only at positions that are relatively far from the region around the position at which the measurement-object gas is blown against the side portion 146a in a direction perpendicular to the side portion 146a. Accordingly, the flow of the measurement-object gas that tries to flow out through the horizontal holes 147b is not greatly impeded, and the responsiveness is not greatly reduced. When the responsiveness greatly varies depending on the relationship between the positions of the outer outlets 147a in the side portion 146a (horizontal holes 147b in this example) and the direction in which the measurement-object gas flows, the responsiveness may be reduced depending on the orientation in which the gas sensor 100 is attached (angle of the outer protective cover 140 around the central axis in the rotational direction). When, for example, the gas sensor 100 is attached to the pipe 20 in such an orientation that the measurement-object gas flows in the direction of arrow D1, the responsiveness tends to decrease. In contrast, in the gas sensor 100 according to the present embodiment, since the outer outlets 147a are not formed in the side portion 146a, the influence of the orientation in which the gas sensor 100 is attached on the responsiveness can be reduced. The influence of the orientation in which the gas sensor 100 is attached on the responsiveness is referred to as angular dependence. In the gas sensor 100 according to the present embodiment, the angular dependence can be reduced because the outer outlets 147a are not formed in the side portion 146a.

In the gas sensor 100 according to the present embodiment described in detail above, since the minimum path length P from the outer inlets 144a to the gas inlet 111 is 11.0 mm or less, the responsiveness in gas concentration detection is increased. In addition, since the minimum path length P is 5.0 mm or more, the occurrence of problems due to insufficient minimum path length P can be reduced. In addition, since the cross-sectional area ratio S1/S2 is more than 2.0 and 5.0 or less, the responsiveness in gas concentration detection is increased. In addition, since no outer outlets 147a are arranged in the side portion 146a, the influence of the orientation in which the gas sensor 100 is attached on the responsiveness can be reduced. As a result, the above-described effect that the responsiveness in gas concentration detection increases can be easily obtained irrespective of the attachment orientation.

In addition, in the gas sensor 100, the element-chamber inlet 127 is formed in the inner protective cover 130 so that the element-side opening 129 opens in the forward direction. Therefore, the measurement-object gas that has flowed out of the element-side opening 129 is not blown against a surface of the sensor element 110 (surface other than the gas inlet 111) in a direction perpendicular to the surface of the sensor element 110, nor does it flow a long distance along the surface of the sensor element 110 before reaching the gas inlet 111. Accordingly, cooling of the sensor element 110 can be reduced. Cooling of the sensor element 110 is reduced by adjusting the direction in which the element-side opening 129 opens, and not by reducing the flow rate and flow velocity of the measurement-object gas inside the inner protective cover 130. Therefore, the amount of reduction in the responsiveness in gas concentration detection can be reduced. As a result, the sensor element 110 has quick responsiveness and high heat retaining properties at the same time.

The present invention is not limited to the above-described embodiment in any way, and can be implemented in various forms within the technical scope of the present invention.

For example, the shape of the protective cover 120 is not limited to that in the above-described embodiment. The shape of the protective cover 120 and the shapes, numbers, arrangements, etc., of the element-chamber inlet 127, the element-chamber outlet 138a, the outer inlets 144a, and the outer outlets 147a may be changed as appropriate. For example, although the element-chamber inlet 127 is formed as a gap between the first member 131 and the second member 135, the element-chamber inlet is not limited to this, and may be formed in any shape as long as the element-chamber inlet serves as an entrance to the sensor element chamber 124. For example, the element-chamber inlet may be a through hole formed in the inner protective cover 130. Also when the element-chamber inlet is a through hole, the element-chamber inlet may serve as a flow channel extending from the back-end side toward the front-end side of the sensor element 110. For example, the element-chamber inlet may be a vertical hole or a hole oblique to the vertical direction in FIG. 3. Also, the element-side opening 129 may be formed so as to open in the forward direction. The element-chamber inlet 127 is not limited to one in number, and may instead be provided in a plurality. The element-chamber outlet 138a, the outer inlets 144a, and the outer outlets 147a are not limited to holes, and may instead be gaps between members that constitute the protective cover 120. These components may be provided in any number as long as they are provided. Although the outer inlets 144a include the horizontal holes 144b and the vertical holes 144c, the outer inlets 144a may include only the horizontal holes 144b or only the vertical holes 144c. Also, corner holes may be formed at the corner between the side portion 143a and the step portion 143b in place of, or in addition to, the horizontal holes 144b and the vertical holes 144c. Similarly, the element-chamber inlet 127, the element-chamber outlet 138a, and the outer outlets 147a may include one or more of a horizontal hole, a vertical hole, and a corner hole. However, as described above, the outer outlets 147a preferably do not include horizontal holes. In other words, the outer outlets 147a are preferably not arranged in the side portion 146a.

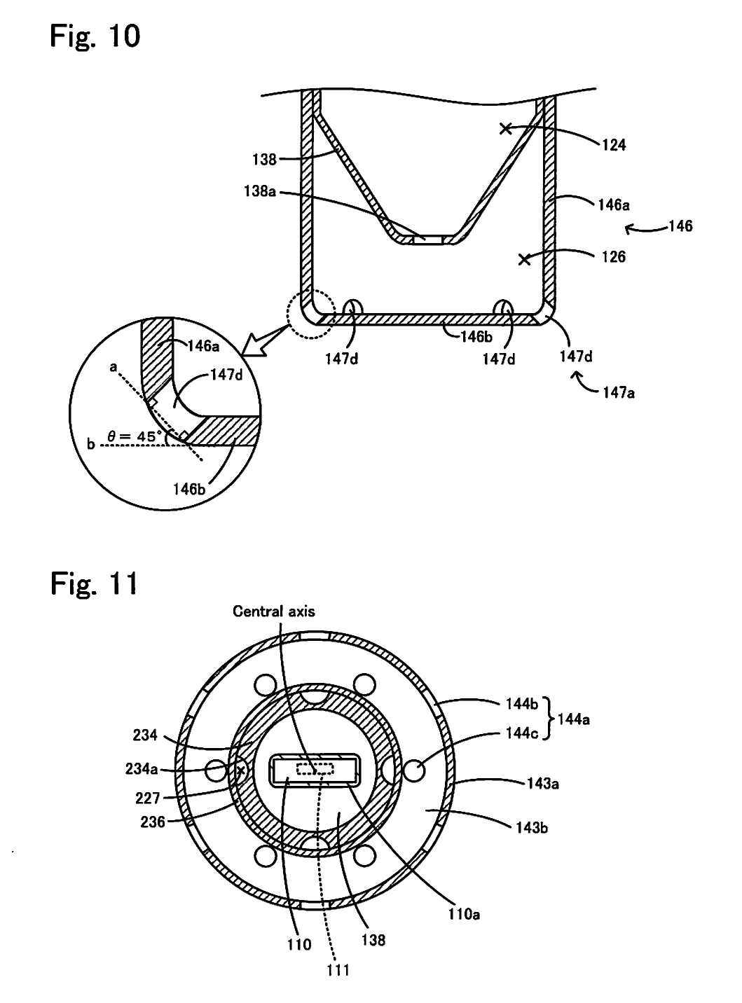

Examples of corner holes will now be described. FIG. 10 is a sectional view illustrating the case in which the outer outlets 147a include a plurality of corner holes 147d. As illustrated in FIG. 10, the outer outlets 147a formed in the front end portion 146 include the corner holes 147d formed at the corner between the side portion 146a and the bottom portion 146b in place of the vertical holes 147c. Six corner holes 147d (only four corner holes 147d are illustrated in FIG. 10) are arranged at equal intervals in the circumferential direction of the outer protective cover 140. The corner holes 147d may be formed so that the angle θ between the outer openings of the corner holes 147d (straight line a in the enlarged view at the lower left in FIG. 10) and the bottom surface (lower surface) of the bottom portion 146b (straight line b in the enlarged view at the lower left in FIG. 10) is in the range of 10° to 80°. In FIG. 10, the angle θ is 45°. Also when corner holes are formed at the corner between the side portion 143a and the step portion 143b in the above-described embodiment, the angle θ between the outer openings of the corner holes and the bottom surface (lower surface) of the bottom portion 146b may be in the range of 10° to 80°.

In the above-described embodiment, the protruding portions 136a are formed on the inner peripheral surface of the second cylindrical portion 136. However, the protruding portions 136a are not limited to this as long as a plurality of protruding portions are formed on at least one of the outer peripheral surface of the first cylindrical portion 134 and the inner peripheral surface of the second cylindrical portion 136 so as to protrude toward and be in contact with the other. In addition, in the above-described embodiment, as illustrated in FIGS. 3 and 4, the outer peripheral surface of the second cylindrical portion 136 is inwardly recessed at the locations where the protruding portions 136a are formed. However, it is not necessary that the outer peripheral surface of the second cylindrical portion 136 be recessed. The shape of the protruding portions 136a is not limited to a hemispherical shape, and may be any shape. Note that it is not necessary that the protruding portions 136a be formed on the outer peripheral surface of the first cylindrical portion 134 or the inner peripheral surface of the second cylindrical portion 136.

In the above-described embodiment, the element-chamber inlet 127 is a tubular gap between the outer peripheral surface of the first cylindrical portion 134 and the inner peripheral surface of the second cylindrical portion 136. However, the element-chamber inlet 127 is not limited to this. For example, a recess (groove) may be formed in at least one of the outer peripheral surface of the first cylindrical portion and the inner peripheral surface of the second cylindrical portion, and the element-chamber inlet may be formed as the gap defined by the recess between the first cylindrical portion and the second cylindrical portion. FIG. 11 is a sectional view illustrating element-chamber inlets 227 according to a modification. Referring to FIG. 11, the outer peripheral surface of a first cylindrical portion 234 and the inner peripheral surface of a second cylindrical portion 236 are in contact with each other, and a plurality of recesses 234a (four recesses 234a in FIG. 11) are formed in the outer peripheral surface of the first cylindrical portion 234 at equal intervals therebetween. The gaps between the inner peripheral surface of the second cylindrical portion 236 and the recesses 234a serve as element-chamber inlets 227.

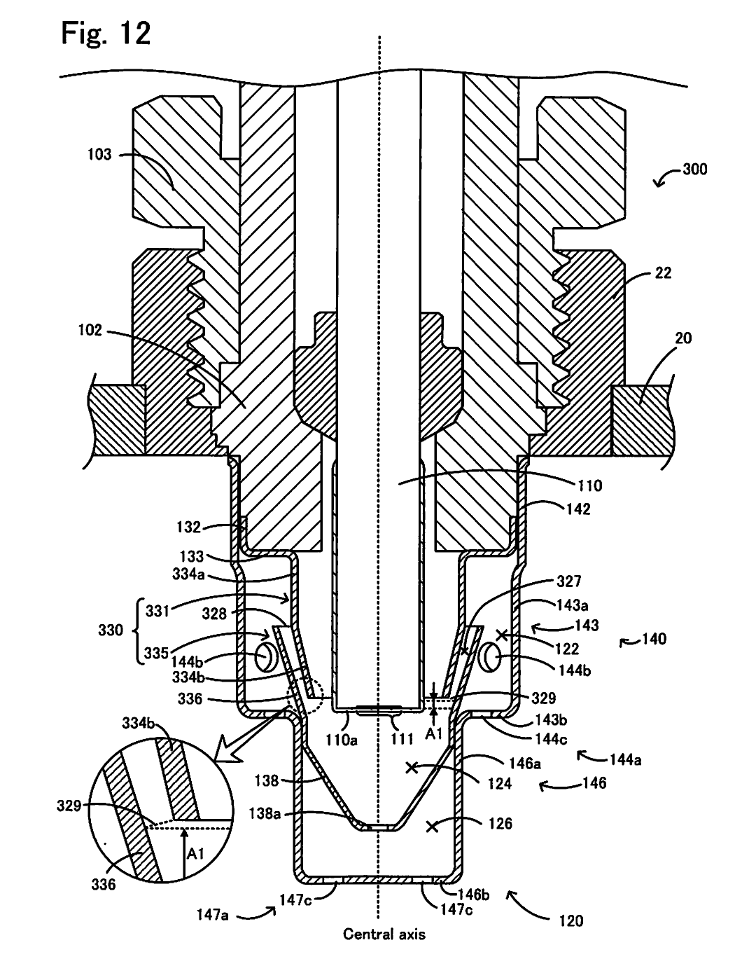

In the above-described embodiment, the element-chamber inlet 127 is a flow channel parallel to the front-back direction of the sensor element 110 (vertical direction in FIG. 3). However, the element-chamber inlet is not limited to this. For example, the element-chamber inlet may instead be formed as a flow channel that is oblique to the front-back direction so that the flow channel becomes closer to the sensor element 110 with increasing distance in the back-to-front direction of the sensor element 110. FIG. 12 is a vertical sectional view of a gas sensor 300 according to a modification in this case. In FIG. 12, components that are the same as those of the gas sensor 100 are denoted by the same reference numerals, and detailed description thereof is omitted. As illustrated in FIG. 12, the gas sensor 300 includes an inner protective cover 330 in place of the inner protective cover 130. The inner protective cover 330 includes a first member 331 and a second member 335. In place of the first cylindrical portion 134 of the first member 131, the first member 331 includes a body portion 334a having a cylindrical shape and a first cylindrical portion 334b having a diameter that decreases with increasing distance in the back-to-front direction of the sensor element 110. The back end of the first cylindrical portion 334b in the front-back direction of the sensor element 110 is connected to the body portion 334a. In place of the second cylindrical portion 136 and the connection portion 137 included in the second member 135, the second member 335 includes a second cylindrical portion 336 having a diameter that decreases with increasing distance in the back-to-front direction of the sensor element 110. The second cylindrical portion 336 is connected to the front end portion 138. The outer peripheral surface of the first cylindrical portion 334b and the inner peripheral surface of the second cylindrical portion 336 are not in contact with each other, and the gap formed therebetween serves as an element-chamber inlet 327. The element-chamber inlet 327 has an outer opening 328, which is an opening adjacent to the first gas chamber 122, and an element-side opening 329, which is an opening adjacent to the sensor element chamber 124. The first cylindrical portion 334b and the second cylindrical portion 336 are shaped so that the element-chamber inlet 327 serves as a flow channel that is oblique to the front-back direction so that the flow channel becomes closer to the sensor element 110 (closer to the central axis of the inner protective cover 330) with increasing distance in the back-to-front direction of the sensor element 110. Similarly, the element-side opening 329 opens obliquely to the front-back direction so as to become closer to the sensor element 110 with increasing distance in the back-to-front direction of the sensor element 110 (see the enlarged view in FIG. 12). When the element-chamber inlet 327 is an oblique flow channel or when the element-side opening 329 is oblique as described above, the measurement-object gas flows into the sensor element chamber 124 through the element-side opening 329 in a direction oblique to the front-back direction of the sensor element 110. Accordingly, an effect similar to that of the element-chamber inlet 127 and the element-side opening 129 according to the above-described embodiment can be obtained. In other words, the measurement-object gas is not blown against the surface of the sensor element 110 (surface other than the gas inlet 111) in a direction perpendicular to the surface of the sensor element 110, nor does it flow a long distance along the surface of the sensor element 110 before reaching the gas inlet 111. Accordingly, cooling of the sensor element 110 can be reduced. In FIG. 12, the element-chamber inlet 327 has a width that decreases with increasing distance in the back-to-front direction of the sensor element 110. Therefore, the opening area of the element-side opening 329 is smaller than that of the outer opening 328. In other words, the element-chamber inlet 327 is formed so that the distance A4 described above with reference to FIG. 7 is smaller than the distance A5. Accordingly, when the measurement-object gas enters through the outer opening 328 and flows out through the element-side opening 329, the measurement-object gas flows out at a flow velocity higher than that at which the measurement-object gas enters. Therefore, the responsiveness in gas concentration detection can be increased. In FIG. 12, the element-chamber inlet 327 serves as a flow channel that is oblique to the front-back direction of the sensor element 110, the element-side opening 329 opens obliquely to the front-back direction of the sensor element 110, and the opening area of the element-side opening 329 is smaller than that of the outer opening 328. However, one or more of these three features may be omitted. In the gas sensor 300 according to the modification, as illustrated in FIG. 12, the distance A1 is the distance from the gas inlet 111 to the bottom end of the element-side opening 329 in the vertical direction. Also in the gas sensor 300 illustrated in FIG. 12, when the minimum path length P is 5.0 mm or more and 11.0 mm or less, an effect similar to that of the above-described embodiment can be obtained.

In the above-described embodiment, the element-side opening 129 opens in the forward direction. However, the element-side opening 129 is not limited to this, and may instead open in the sensor element chamber 124 in a direction perpendicular to the forward direction. In addition, in the above-described embodiment, the element-chamber inlet 127 is a flow channel that is parallel to the front-back direction of the sensor element 110. However, the element-chamber inlet 127 is not limited to this. For example, the element-chamber inlet 127 may instead be a flow channel that is perpendicular to the forward direction.

In the above-described embodiment, the first gas chamber 122 is the only flow channel for the measurement-object gas between the element-chamber inlet 127 and the outer inlets 144a. However, the first gas chamber 122 is not limited to this as long as the first gas chamber 122 is at least a portion of the flow channel for the measurement-object gas between the element-chamber inlet 127 and the outer inlets 144a. For example, the protective cover 120 may include, in addition to the inner protective cover 130 and the outer protective cover 140, an intermediate protective cover disposed between the inner protective cover 130 and the outer protective cover 140, and the flow channel for the measurement-object gas between the element-chamber inlet 127 and the outer inlets 144a may include a plurality of gas chambers. Similarly, in the above-described embodiment, the second gas chamber 126 is the only flow channel for the measurement-object gas between the element-chamber outlet 138a and the outer outlets 147a. However, the second gas chamber 126 is not limited to this as long as the second gas chamber 126 is at least a portion of the flow channel for the measurement-object gas between the element-chamber outlet 138a and the outer outlets 147a.

In the above-described embodiment, the gas inlet 111 opens in the front end face of the sensor element 110 (lower surface of the sensor element 110 in FIG. 3). However, the gas inlet 111 is not limited to this. For example, the gas inlet 111 may open in a side surface of the sensor element 110 (upper, lower, left, or right surface of the sensor element 110 in FIG. 4).

In the above-described embodiment, the sensor element 110 includes the porous protective layer 110a. However, it is not necessary that the sensor element 110 include the porous protective layer 110a.

### EXAMPLES

Examples of gas sensors that were actually manufactured will now be described. Experimental Examples 3 to 5 correspond to examples of the present invention, and Experimental Examples 1 and 2 correspond to comparative examples. The present invention is not limited to the following examples.

### Experimental Example 1

A gas sensor 100 according to Experimental Example 1 was similar to the gas sensor 100 illustrated in FIGS. 3 to 7 except that, as illustrated in FIGS. 8 and 9, the outer outlets 147a included the three horizontal holes 147b formed in the side portion 146a and the three vertical holes 147c. The first member 131 of the inner protective cover 130 had a thickness of 0.3 mm and an axial length of 10.2 mm. The large-diameter portion 132 had an axial length of 1.8 mm and an outer diameter of 14.4 mm, and the first cylindrical portion 134 had an axial length of 8.4 mm and an outer diameter of 7.7 mm. The second member 135 had a thickness of 0.3 mm and an axial length of 11.5 mm. The second cylindrical portion 136 had an axial length of 4.5 mm and an inner diameter of 9.7 mm, and the front end portion 138 had an axial length of 4.9 mm. The bottom surface of the front end portion 138 had a diameter of 3.0 mm. With regard to the element-chamber inlet 127, the distance A1 was 0.59 mm, the distance A2 was 1.7 mm, the distance A3 was 3.1 mm, the distances A4, A5, and A7 were 1.0 mm, the distance A6 was 2.05 mm, and the length L was 4 mm. The element-chamber outlet 138a had a diameter of 1.5 mm. The outer protective cover 140 had a thickness of 0.4 mm and an axial length of 24.35 mm. The large-diameter portion 142 had an axial length of 5.85 mm and an outer diameter of 15.2 mm. The body portion 143 had an axial length of 8.9 mm (axial length from the upper end of the body portion 143 to the upper surface of the step portion 143b was 8.5 mm). The body portion 143 had an outer diameter of 14.6 mm. The front end portion 146 had an axial length of 9.6 mm and an outer diameter of 8.7 mm. The outer inlets 144a included six horizontal holes 144b having a diameter of 1 mm and six vertical holes 144c having a diameter of 1 mm. The horizontal holes 144b and the vertical holes 144c were alternately arranged at equal intervals (the adjacent holes form an angle of 30°). The outer outlets 147a included three horizontal holes 147b having a diameter of 1 mm and three vertical holes 147c having a diameter of 1 mm. The horizontal holes 147b and the vertical holes 147c were alternately arranged at equal intervals (the adjacent holes form an angle of 60°). The material of the protective cover 120 was SUS301S. The sensor element 110 of the gas sensor 100 had a width (length in the left-right direction in FIG. 4) of 4 mm and a thickness (length in the vertical direction in FIG. 4) of 1.5 mm. The porous protective layer 110a was an alumina porous body having a thickness of 400 μm. The minimum path length P was 11.4 mm. The total cross-sectional area S1 was 9.42 mm². The total cross-sectional area S2 was 4.71 mm². The cross-sectional area ratio S1/S2 was 2.00.

### Experimental Example 2

A gas sensor 100 according to Experimental Example 2 was similar to the gas sensor 100 according to Experimental Example 1 except that the inner diameter of the first cylindrical portion 134 of the first member 131 was 7.88 mm, which was greater than that in Experimental Example 1. In Experimental Example 2, the distances A4, A5, and A7 were 0.61 mm, the distance A2 was 2.1 mm, the minimum path length P was 11.7 mm, the total cross-sectional area S1 was 9.42 mm², the total cross-sectional area S2 was 4.71 mm², and the cross-sectional area ratio S1/S2 was 2.00.

### Experimental Example 3

A gas sensor 100 according to Experimental Example 3 was the gas sensor 100 illustrated in FIGS. 3 to 7. In Experimental Example 3, the outer outlets 147a did not include the horizontal holes 147b, and the diameter of the vertical holes 147c was 1 mm as in Experimental Example 1. The horizontal holes 144b had a diameter of 1.5 mm, and were shifted backward so that the distance A3 was 0.84 mm. Other dimensions were the same as those in Experimental Example 2. The minimum path length P was 10.0 mm, the total cross-sectional area S1 was 15.32 mm², the total cross-sectional area S2 was 4.71 mm², and the cross-sectional area ratio S1/S2 was 3.25.

### Experimental Example 4

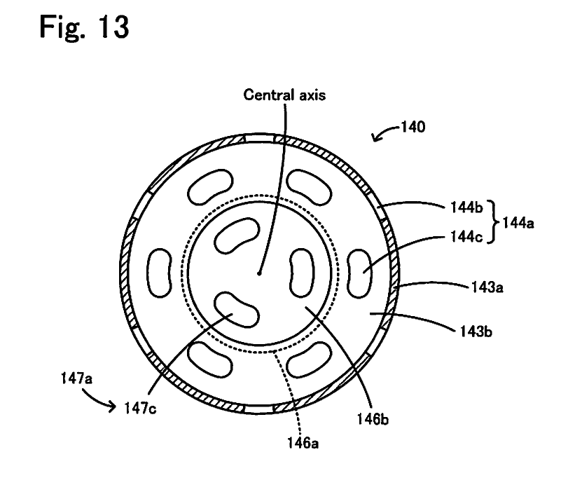

A gas sensor 100 according to Experimental Example 4 was the same as the gas sensor 100 of Experimental Example 3 except that the cross-sectional area of the vertical holes 144c and the cross-sectional area of the three vertical holes 147c were increased. More specifically, as illustrated in FIG. 13, the vertical holes 144c and the vertical holes 147c were formed in an arc shape that extends in the circumferential direction of the outer protective cover 140, so that the cross-sectional areas thereof were increased. The vertical holes 144c and the vertical holes 147c were formed in an arc shape having a width of 1 mm. The six vertical holes 144c had a cross-sectional area of 2.4 mm². The three vertical holes 147c also had a cross-sectional area of 2.4 mm². The minimum path length P was 10.0 mm, the total cross-sectional area S1 was 25.03 mm², the total cross-sectional area S2 was 7.21 mm², and the cross-sectional area ratio S1/S2 was 3.47.

### Experimental Example 5

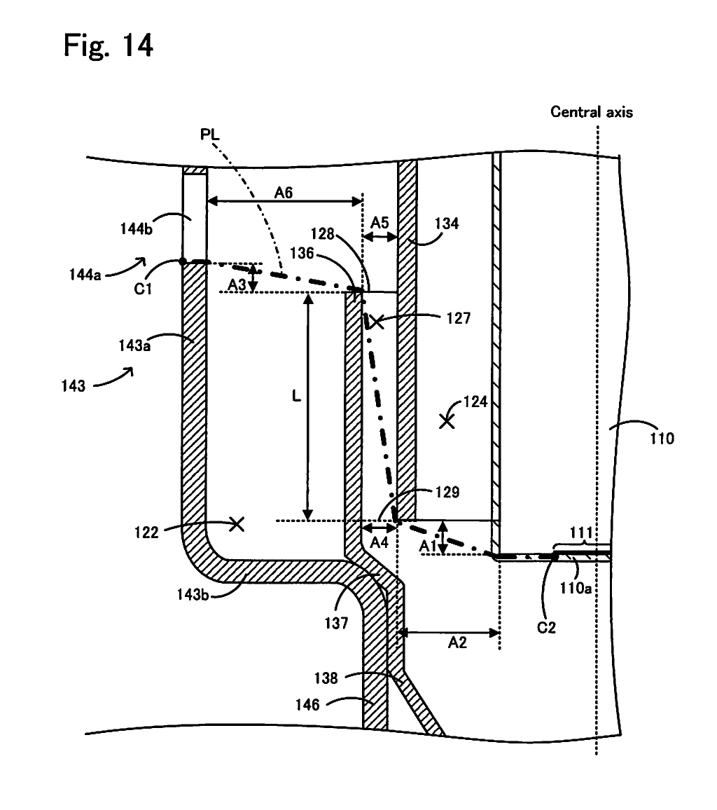

A gas sensor 100 according to Experimental Example 5 was the same as the gas sensor 100 according to Experimental Example 3 except that the horizontal holes 144b were closer to the back end than the outer opening 128, as illustrated in FIG. 14, and the distance A3 was −0.16 mm. The minimum path length P was 9.9 mm. Referring to FIG. 14, in the gas sensor 100 according to Experimental Example 5, the minimum path length P was the length of the shortest path (broken line PL) from an end portion C1 (lower end portion in FIG. 14) of the outer opening of the horizontal hole 144b, the end portion C1 being closest to the outer opening 128, to an end portion C2 (left end portion in FIG. 14) of the outer opening of the gas inlet 111. The total cross-sectional area S1 was 15.32 mm², the total cross-sectional area S2 was 4.71 mm², and the cross-sectional area ratio S1/S2 was 3.25.

[Evaluation of Angular Dependence]

The influence of the attachment orientation of each of the gas sensors according to Experimental Examples 1 to 5 on the responsiveness (angular dependence) was evaluated. First, the gas sensor according to Experimental Example 1 was attached to a pipe in a manner illustrated in FIGS. 1 and 2. The attachment orientation of the gas sensor according to Experimental Example 1 was such that the measurement-object gas flowed through the pipe in the direction of arrow D1 in FIG. 8. Gas obtained by mixing oxygen with atmospheric air to adjust the oxygen concentration was used as the measurement-object gas. The measurement-object gas was caused to flow through the pipe at a flow velocity of V=8 m/s. The oxygen concentration of the measurement-object gas that flowed through the pipe was changed from 22.9% to 20.2%, and a change in the output of the sensor element over time was measured. The output value of the sensor element immediately before the change in oxygen concentration was defined as 0%, and the output value of the sensor element at the time when the output of the sensor element became stable after the change in oxygen concentration was defined as 100%. The time from when the output value exceeded 10% to when the output value exceeded 90% was defined as the response time (sec) in gas concentration detection. The shorter the response time, the higher the responsiveness in gas concentration detection. The attachment orientation of the gas sensor according to Experimental Example 1 was changed to multiple orientations, and the response time was measured for each attachment orientation. More specifically, when the attachment orientation for causing the measurement-object gas to flow in the direction of arrow D1 in FIG. 8 was defined as 0°, the attachment orientation of the gas sensor was changed from 0° to 360° in steps of 30° by rotating the gas sensor around the central axis of the outer protective cover 140, and the response time was measured for each attachment orientation. The attachment orientation of the gas sensor is the same for 0° and 360°. Each of the gas sensors according to Experimental Examples 2 to 5 was also attached in different orientations, and the response time was measured for each attachment orientation. In the gas sensor according to Experimental Example 2, similar to Experimental Example 1, the attachment orientation for causing the measurement-object gas to flow in the direction of arrow D1 in FIG. 8 was defined as 0°. In Experimental Examples 3 to 5, the attachment orientation for causing the measurement-object gas to flow from the upper left toward the lower right in FIG. 4 in a direction parallel to the direction in which the upper left horizontal hole 144b in FIG. 4 opens was defined as 0°.

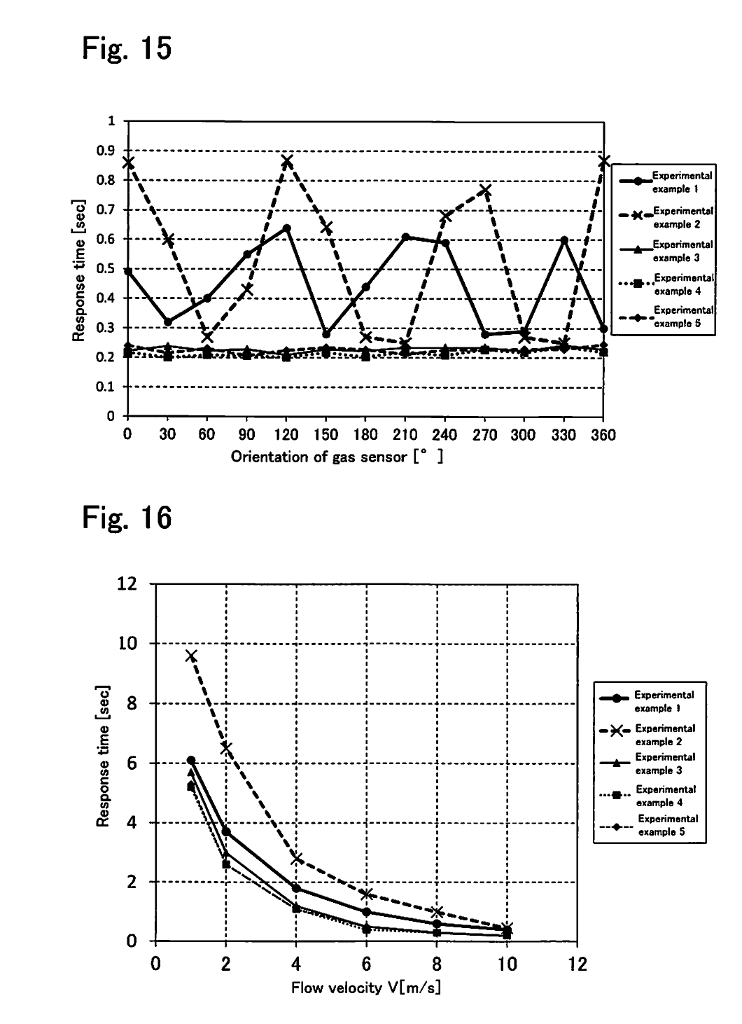

FIG. 15 is a graph showing the angular dependence of the response time of each of the gas sensors according to Experimental Examples 1 to 5. As is clear from FIG. 15, in Experimental Examples 1 and 2, the response time greatly varies depending on the attachment orientation of the gas sensor. Thus, the response time had a high angular dependence. More specifically, in Experimental Examples 1 and 2, the response time periodically increased at intervals of substantially 120°. In Experimental Examples 1 and 2, the outer outlets 147a included three horizontal holes 147b formed in the side portion 146a, and the horizontal holes 147b were arranged at equal intervals (120°) around the central axis of the outer protective cover 140. Therefore, when the attachment orientation was 0°, 120°, 240°, or 360°, one of the horizontal holes 147b opened parallel to, and toward the upstream side of, the direction in which the measurement-object gas flowed. Accordingly, in Experimental Examples 1 and 2, when the attachment orientation was 0°, 120°, 240°, or 360°, the flow of the measurement-object gas that tried to flow out of the outer protective cover 140 through this horizontal hole 147b was impeded by the measurement-object gas that flowed around this horizontal hole 147b, and the responsiveness tended to decrease as a result. In contrast, in Experimental Examples 3 to 5, as is clear from FIG. 15, variations in the response time depending on the attachment orientation were significantly smaller than those in Experimental Examples 1 and 2. Thus, the angular dependence was low. This is probably because no outer outlets 147a were formed in the side portion 146a in Experimental Examples 3 to 5.

[Evaluation of Responsiveness]

For each of the gas sensors according to Experimental Examples 1 to 5, the flow velocity V of the measurement-object gas that flowed through the pipe was set to 1, 2, 4, 6, 8, and 10 m/s, and the response time [sec] was measured for each flow velocity V. The response time was measured in a way similar to that for measuring the response time to evaluate the above-described angular dependence. When the flow velocity was V=8 m/s, as in the above-described case of evaluating the angular dependence, the attachment orientation was changed from 0° to 360°, and the response time was measured multiple times for each attachment orientation. In addition, the oxygen concentration in the measurement-object gas that flowed through the pipe was changed from 20.2% to 22.9% (change opposite to that in the evaluation of the angular dependence). Also in this case, the attachment orientation was similarly changed from 0° to 360°, and the response time was measured multiple times for each attachment orientation. The average of all of the response times was determined as the response time for the flow velocity V=8 m/s. In other cases (flow velocity V=1, 2, 4, 6, and 10 m/s), the attachment orientation was not changed. The response time was measured after the oxygen concentration in the measurement-object gas that flowed through the pipe was reduced (from 22.9% to 20.2%) and increased (from 20.2% to 22.9%), and the average of the response times was determined as the response time for each flow velocity V. The attachment orientation was set to 0° in Experimental Examples 1 and 2, and to 180° in Experimental Examples 3 to 5.

Table 1 shows the diameters and numbers of outer inlets and outer outlets in the outer protective cover, the minimum path length P, the total cross-sectional areas S1 and S2, the cross-sectional area ratio S1/S2, and the response time for each flow velocity V in Experimental Examples 1 to 5. FIG. 16 is a graph showing the relationship between the flow velocity V and the response time in Experimental Examples 1 to 5.

TABLE 1

| | | | | Total | Total | | |
|---|---|---|---|---|---|---|---|
| | | | Minimum | cross- | cross- | Cross- | Response time |
| | | | path | sectional | sectional | sectional | (Flow velocity |
| | Outer protective cover | | length P | area S1 | area S2 | area ratio | 1 m/s) |
| | Outer inlet | Outer outlet | [mm] | [mm²] | [mm²] | S1/S2 | [sec] |
| Experimental | Diameter of 1 mm × 6 | Diameter of 1 mm × 3 | 11.4 | 9.42 | 4.71 | 2.00 | 6.1 |
| example 1 | (horizontal hole) | (horizontal hole) | | | | | |
| | Diameter of 1 mm × 6 | Diameter of 1 mm × 3 | | | | | |
| | (vertical hole) | (vertical hole) | | | | | |
| Experimental | Diameter of 1 mm × 6 | Diameter of 1 mm × 3 | 11.7 | 9.42 | 4.71 | 2.00 | 9.6 |
| example 2 | (horizontal hole) | (horizontal hole) | | | | | |
| | Diameter of 1 mm × 6 | Diameter of 1 mm × 3 | | | | | |
| | (vertical hole) | (vertical hole) | | | | | |
| Experimental | Diameter of 1.5 mm × 6 | Diameter of 1 mm × 6 | 10.0 | 15.32 | 4.71 | 3.25 | 5.7 |
| example 3 | (horizontal hole) | (vertical hole) | | | | | |
| | Diameter of 1 mm × 6 | | | | | | |
| | (vertical hole) | | | | | | |
| Experimental | Diameter of 1.5 mm × 6 | 2.4 mm² × 3 | 10.0 | 25.03 | 7.21 | 3.47 | 5.2 |
| example 4 | (horizontal hole) | (vertical hole) | | | | | |
| | 2.4 mm² × 6 | | | | | | |
| | (vertical hole) | | | | | | |
| Experimental | Diameter of 1.5 mm × 6 | Diameter of 1 mm × 6 | 9.9 | 15.32 | 4.71 | 3.25 | 5.3 |
| example 5 | (horizontal hole) | (vertical hole) | | | | | |
| | Diameter of 1 mm × 6 | | | | | | |
| | (vertical hole) | | | | | | |

| | | | | | Response |
|---|---|---|---|---|---|
| | Response time | Response time | Response time | Response time | time |
| | (Flow velocity | (Flow velocity | (Flow velocity | (Flow velocity | (Flow velocity |
| | 2 m/s) | 4 m/s) | 6 m/s) | 8 m/s) | 10 m/s) |
| | [sec] | [sec] | [sec] | [sec] | [sec] |
| Experimental | 3.7 | 1.8 | 1 | 0.6 | 0.4 |
| example 1 | | | | | |
| Experimental | 6.5 | 2.8 | 1.6 | 1 | 0.45 |
| example 2 | | | | | |
| Experimental | 3 | 1.2 | 0.5 | 0.3 | 0.2 |
| example 3 | | | | | |
| Experimental | 2.6 | 1.1 | 0.4 | 0.3 | 0.2 |
| example 4 | | | | | |
| Experimental | 2.6 | 1.1 | 0.5 | 0.3 | 0.2 |
| example 5 | | | | | |

Table 1 and FIG. 16 show that, in each of Experimental Examples 1 to 5, the response time increased as the flow velocity V decreased. At each flow velocity V, the response times in Experimental Examples 3 to 5 were shorter than those in Experimental Examples 1 and 2. More specifically, the response times in Experimental Examples 3 to 5, in which the minimum path length P was 11.0 mm or less and the cross-sectional area ratio S1/S2 was more than 2.0, were shorter than those in Experimental Examples 1 and 2, in which the minimum path length P was more than 11.0 mm and the cross-sectional area ratio S1/S2 was 2.0 or less. In Experimental Examples 1 to 5, the response time decreased as the minimum path length P decreased. A comparison between Experimental Examples 3 and 5, which had the same cross-sectional area ratio S1/S2 and different minimum path lengths P, shows that the minimum path length P is preferably less than 10.0 mm. In Experimental Examples 1 to 5, the response time decreased as the cross-sectional area ratio S1/S2 increased. A comparison between Experimental Examples 3 and 4, which had the same minimum path length P and different cross-sectional area ratios S1/S2, shows that the cross-sectional area ratio S1/S2 is preferably 3.4 or more. A comparison between Experimental Examples 2 to 5, in which the first cylindrical portions 134 of the first members 131 had the same inner diameter and in which the protective covers 120 had relatively similar shapes, shows that the differences between the response time of Experimental Example 2 and the response times of Experimental Examples 3 to 5 increase as the flow velocity V decreases. This shows that when, in particular, the flow velocity V is low (4 m/s or less), the response-time reducing effect obtained by setting the minimum path length P to 11.0 mm or less or by setting the cross-sectional area ratio S1/S2 to more than 2.0 probably increases.

The present application claims priority from Japanese Patent Application No. 2016-121007, filed on Jun. 17, 2016, the entire contents of which are incorporated herein by reference.

What is claimed is:

1. A gas sensor comprising:
a sensor element having a gas inlet through which measurement-object gas is introduced and capable of detecting a concentration of a predetermined gas in the measurement-object gas that flows into the sensor element through the gas inlet;
an inner protective cover that has a sensor element chamber thereinside and in which one or more element-chamber inlet and one or more element-chamber outlet are arranged, the sensor element chamber accommodating a front end of the sensor element and the gas inlet, the element-chamber inlet being an entrance to the sensor element chamber, and the element-chamber outlet being an exit from the sensor element chamber; and
an outer protective cover that is disposed outside the inner protective cover and in which one or more outer inlet and one or more outer outlet are arranged, the outer inlet being an entrance from outside for the measurement-object gas, and the outer outlet being an exit to the outside for the measurement-object gas,
wherein the outer protective cover and the inner protective cover form a first gas chamber and a second gas chamber as spaces therebetween, the first gas chamber being at least a portion of a flow channel for the measurement-object gas between the outer inlet and the element-chamber inlet, and the second gas chamber being at least a portion of a flow channel for the measurement-object gas between the outer outlet and the element-chamber outlet and not being directly connected to the first gas chamber, and
a minimum path length P from the outer inlet to the gas inlet is 5.0 mm or more and 11.0 mm or less.

2. The gas sensor according to claim 1, wherein the minimum path length P is 10.0 mm or less.

3. The gas sensor according to claim 1, wherein a cross-sectional area ratio S1/S2, which is a ratio of a total cross-sectional area S1 [mm²] of the outer inlet to a total cross-sectional area S2 [mm²] of the outer outlet, is more than 2.0 and 5.0 or less.

4. The gas sensor according to claim 3, wherein the cross-sectional area ratio S1/S2 is 3 or more.

5. The gas sensor according to claim 3, wherein the total cross-sectional area S1 is 10 mm² or more and 30 mm² or less.

6. The gas sensor according to claim 3, wherein the total cross-sectional area S2 is 2 mm² or more and 10 mm² or less.

7. The gas sensor according to claim 1, wherein the outer protective cover has a cylindrical shape and includes a side portion and a bottom portion, and
the outer outlet is not arranged in the side portion of the outer protective cover.

8. The gas sensor according to claim 1, wherein the outer protective cover includes a body portion, which has a cylindrical shape and in which the outer inlet is arranged, and a front end portion, which has a cylindrical shape with a bottom and an inner diameter smaller than an inner diameter of the body portion and in which the outer outlet is arranged, the front end portion being located in front of the body portion in a forward direction, which is a direction from a back end toward the front end of the sensor element, and
the outer protective cover and the inner protective cover form the first gas chamber as a space between the body portion of the outer protective cover and the inner protective cover, and the second gas chamber as a space between the front end portion of the outer protective cover and the inner protective cover.

9. The gas sensor according to claim 1, wherein the element-chamber inlet is formed in the inner protective cover so that an element-side opening of the element-chamber inlet that is close to the sensor element chamber opens in a forward direction, which is a direction from a back end toward the front end of the sensor element.

10. The gas sensor according to claim 1, wherein the inner protective cover includes a first member and a second member, and
the element-chamber inlet is formed as a gap between the first member and the second member.

11. The gas sensor according to claim 10, wherein the first member includes a first cylindrical portion that surrounds the sensor element,
the second member includes a second cylindrical portion having a diameter greater than a diameter of the first cylindrical portion, and
the element-chamber inlet is a tubular gap between an outer peripheral surface of the first cylindrical portion and an inner peripheral surface of the second cylindrical portion.
